(19) United States
(12) Patent Application Publication
(10) Pub. No.: US 2025/0269108 A1
(43) Pub. Date: Aug. 28, 2025

(54) SYSTEM AND METHOD FOR ADMINISTERING A PHARMACEUTICAL COMPOSITION WITH AN INTRAVENOUS TUBING SET

(71) Applicant: ImmunityBio, Inc., Culver City, CA (US)

(72) Inventors: Kathleen Adlard, Culver City, CA (US); Azita Nourani, Orange, CA (US)

(21) Appl. No.: 19/064,420

(22) Filed: Feb. 26, 2025

Related U.S. Application Data
(60) Provisional application No. 63/558,017, filed on Feb. 26, 2024.

(51) Int. Cl. A61M 5/14; A61M 39/08; A61M 39/28

(52) U.S. Cl. CPC A61M 5/1408; A61M 39/08; A61M 39/28

(57) ABSTRACT

An intravenous tubing set for use in a system and method for administering a pharmaceutical composition includes a first spike on a first line, optionally installed via a coupler, for a pharmaceutical composition and a second spike on a second line for a solution. The first line and the second line are in fluid communication with a dual port chamber, which is in fluid communication with a coupler that is able to engage an instrument attached to a patient at an intravenous site. Where the pharmaceutical composition is a dose separated into multiple portions in multiple infusion vials, the first spike may be a vented spike that is reusable and insertable into each stopper of the multiple vials. The pharmaceutical composition may be administered using a gravity feed via the intravenous tubing set, without leakage caused by the vented spike dislodging the stopper of the infusion vials.

### CROSS-REFERENCE TO RELATED APPLICATIONS

This application claims the benefit of priority under 35 U.S.C. § 119(e) to U.S. Provisional Application Ser. No. 63/558,017, filed Feb. 26, 2024. The entire disclosure of U.S. Provisional Application Ser. No. 63/558,017 is incorporated herein by reference in its entirety.

### FIELD OF THE DISCLOSURE

The disclosure relates to administering a pharmaceutical composition and, in particular, is directed to a system and method for administering a pharmaceutical composition with an intravenous tubing set.

### BACKGROUND

The background description includes information that may be useful in understanding the presently disclosed subject matter. It is not an admission that any of the information provided herein is prior art or relevant to the present disclosure.

Pharmaceutical compositions may be stored in a container such as an intravenous bag or an infusion vial. One method of administering a pharmaceutical composition includes using a gravity feed, where the container is suspended at a position above an intravenous site and gravity causes the pharmaceutical composition to flow through an intravenous tubing set to the intravenous site. A dosage of the pharmaceutical composition may be administered as a single amount or in multiple amounts. For example, the dosage of the pharmaceutical composition may be separated into multiple amounts stored in different containers, depending on the volume of the container in which the pharmaceutical composition is stored.

In one example, a pharmaceutical composition including at least one drug substance may be stored in an infusion vial with a stopper. Engaging the stopper of the infusion vial with a spike of the intravenous tubing set may cause the stopper to become dislodged. For instance, the stopper may be pushed into the infusion vial while attempting to insert the spike into the stopper. This may cause leakage from the infusion vial, especially when suspended upside down for the gravity feed (i.e., with the stopper facing downward, toward the ground). In addition, this may cause the air filter within the spike to become wet or clogged, which may prevent adequate flow via gravity feed from the infusion vial to the intravenous site. Where the dosage of the pharmaceutical composition is separated into multiple amounts in different containers, the wet or clogged air filter within the spike may result in the need to interchange multiple spikes onto the intravenous tubing set between the infusion vials in which the dosages are stored, to continue administration of the pharmaceutical composition.

### SUMMARY

As such, there exists a need for an improved intravenous tubing set that allows for the administration of a pharmaceutical composition via gravity feed. The intravenous tubing set should be configured for the administration of the pharmaceutical composition in both a single amount stored in a single container and separated into multiple amounts stored in different containers. In some embodiments, the intravenous tubing set should include a spike able to be inserted into a stopper of one infusion vial (or multiple infusion vials, sequentially during administration) without dislodging the stopper to reduce the possibility of leakage from the infusion vial and/or the possibility of the air filter within the spike becoming wet or clogged, thus reducing the need for multiple vented spikes being interchanged in the intravenous tubing set.

Embodiments of the present disclosure are directed to an intravenous tubing set with a first spike and a second spike. The first spike is insertable into a stopper of one or more infusion vials in which a pharmaceutical composition is stored. The second spike is insertable into a container (i.e., an intravenous bag) containing another pharmaceutical composition. The first spike and the second spike are on respective lines that both are in fluid communication with a dual port chamber. The dual port chamber is further in fluid communication with a patient at an intravenous site. In some instances, the second spike on the second line includes a solution to prime or flush the dual port chamber prior to the administration of the pharmaceutical composition via the first spike on the first line. In other instances, the second spike on the second line includes a solution that is configured to mix with the pharmaceutical composition provided by the first spike on the first line within the dual port chamber. In some embodiments, the first spike is a vented spike, including where the container is a rigid infusion vial, to allow air to flow into the container as the stored pharmaceutical composition flows out and to ensure ambient pressure within the infusion vial. In these embodiments, the second spike is a non-vented spike.

Embodiments of the present disclosure are directed to tubing that extends from the dual port chamber and is in fluid communication with a coupler able to engage an instrument attached to a patient at the intravenous site. The tubing may be separated into sections by a connector. In some instances, the connector is a branched connector, with the dual port chamber in fluid communication with a first branch on a first end and the coupler is in fluid communication with the second end. A second branch on the first end (or along the length of the branched connector) may provide access to the tubing (i.e., for a needleless syringe in communication with the second branch on the first end). Alternatively, the tubing may be a continuous piece of tubing. In some embodiments, the coupler includes a coupling mechanism that is able to engage the instrument at the intravenous site, where the coupling mechanism includes, but is not limited to, is a spinlock or Luer lock fitting.

Embodiments of the present disclosure are directed to clamps on one or more sections of the tubing along the length of the intravenous tubing set. The clamps may be slide clamps or roller clamps.

Embodiments of the present disclosure are directed to the interchanging of infusion vials including the pharmaceutical composition during administration, without need of changing the vented spike able to engage the stopper of the infusion vials. Additional embodiments of the present disclosure are directed to interchanging vented spikes along with the interchanging of infusion vials during administration of the pharmaceutical composition.

A first aspect of the present disclosure is to provide an intravenous tubing set. The intravenous tubing set includes a vented spike operable to be inserted into a stopper of an infusion vial; a first line of tubing having a first end in fluid communication with the vented spike; a non-vented spike operable to be inserted into an intravenous bag of solution; a second line of tubing having a first end in fluid communication with the non-vented spike; and a dual port chamber. A second opposite end of the first line of tubing and a second opposite end of the second line of tubing are each in fluid communication with a first end of the dual port chamber.

The intravenous tubing set of the first aspect may include, optionally, a first clamp operable to control a fluid flow through the first line of tubing; and a second clamp operable to control a fluid flow through the second line of tubing.

The intravenous tubing set of the first aspect may include one or more of the previous embodiments and, optionally, wherein at least one of the first clamp and the second clamp comprises a slide clamp or a roller clamp.

The intravenous tubing set of the first aspect may include one or more of the previous embodiments and, optionally, a third line of tubing in fluid communication with a second end of the dual port chamber and in fluid communication with a coupler, wherein the coupler is able to engage an instrument attached to a patient at the intravenous site.

The intravenous tubing set of the first aspect may include one or more of the previous embodiments and, optionally, a clamp operable to control a fluid flow through the third line of tubing.

The intravenous tubing set of the first aspect may include one or more of the previous embodiments and, optionally, wherein the clamp comprises a roller clamp.

The intravenous tubing set of the first aspect may include one or more of the previous embodiments and, optionally, wherein the coupler includes a coupling mechanism able to engage the instrument attached to the patient at the intravenous site.

The intravenous tubing set of the first aspect may include one or more of the previous embodiments and, optionally, wherein the coupling mechanism comprises a spinlock or a Luer lock.

The intravenous tubing set of the first aspect may include one or more of the previous embodiments and, optionally, wherein the coupler includes a cap operable to cover a distal end of the coupler.

The intravenous tubing set of the first aspect may include one or more of the previous embodiments and, optionally, an additional connector separating the third line of tubing into a first section and a second section. The first section of the third line of tubing is in fluid communication with the second end of the dual port chamber and in fluid communication with a first end of the additional connector. The second section of the third line of tubing is in fluid communication with a second end of the additional connector and in fluid communication with the additional connector.

The intravenous tubing set of the first aspect may include one or more of the previous embodiments and, optionally, wherein a first end of the additional connector includes a first branch and a second branch, and wherein the first section of the third line of tubing is in fluid communication with the first branch of the additional connector.

The intravenous tubing set of the first aspect may include one or more of the previous embodiments and, optionally, wherein the second branch of the additional connector is operable to provide access to the second section of the third line of tubing.

The intravenous tubing set of the first aspect may include one or more of the previous embodiments and, optionally, wherein the second end of the additional connector and the second branch of the first end of the additional connector are substantially collinear along a longitudinal axis through the additional connector, and wherein the first branch of the first end of the additional connector is set at an angle to the longitudinal axis through the additional connector.

The intravenous tubing set of the first aspect may include one or more of the previous embodiments and, optionally, wherein the dual port chamber includes a cap at the first end.

The intravenous tubing set of the first aspect may include one or more of the previous embodiments and, optionally, wherein the first line of tubing and the second line of tubing are separately in fluid communication with the cap.

The intravenous tubing set of the first aspect may include one or more of the previous embodiments and, optionally, wherein the dual port chamber includes a port at a second end opposite to the first end of the dual port chamber.

A second aspect of the present disclosure is to provide an intravenous tubing set. The intravenous tubing set includes tubing comprising a plurality of lines; a non-vented spike coupled to a first line of the plurality of lines; at least a second vented spike coupled to at least a second line of the plurality of lines; and a dual port chamber, wherein the first line and the at least a second line of the plurality of lines are in fluid communication with a stopper at a first end of the dual port chamber.

The intravenous tubing set of the second aspect may include, optionally, wherein the plurality of lines comprises: a first line of tubing having a first end in fluid communication with the at least a second vented spike; and a second line of tubing having a first end in fluid communication with the non-vented spike. A second opposite end of the first line of tubing and a second opposite end of the second line of tubing are each in fluid communication with the first end of the dual port chamber.

A third aspect of the present disclosure is to provide a method for administering a pharmaceutical composition. The method may include, but is not limited to, coupling a first infusion vial and an intravenous bag of solution to an intravenous tubing set to administer a first portion of the pharmaceutical composition. The intravenous tubing set includes a vented spike operable to be inserted into a stopper of the first infusion vial; a first line of tubing having a first end in fluid communication with the vented spike; a non-vented spike operable to be inserted into the intravenous bag of solution; a second line of tubing having a first end in fluid communication with the non-vented spike; and a dual port chamber. A second opposite end of the first line of tubing and a second opposite end of the second line of tubing are in fluid communication with a first end of the dual port chamber. The method may include, but is not limited to, uncoupling the first infusion vial from the intravenous tubing set. The method may include, but is not limited to, coupling a second infusion vial to the intravenous tubing set to administer a second portion of the pharmaceutical composition.

The method of the third aspect may include, optionally, wherein the vented spike is interchangeable with a second vented spike prior to the coupling of the second infusion vial to the intravenous tubing set, wherein the second vented spike is operable to be inserted into a stopper of the second infusion vial.

A fourth aspect of the present disclosure is to provide an intravenous tubing set. The intravenous tubing set includes a vented spike operable to be inserted into a stopper of an infusion vial; a first line of tubing having a first end in fluid communication with the vented spike; a non-vented spike operable to be inserted into an intravenous bag of solution; a second line of tubing having a first end in fluid communication with the non-vented spike; and a dual port chamber. A second opposite end of the first line of tubing and a second opposite end of the second line of tubing are each in fluid communication with a first end of the dual port chamber.

The intravenous tubing set of the fourth aspect may include, optionally, a first clamp operable to control a fluid flow through the first line of tubing; and a second clamp operable to control a fluid flow through the second line of tubing.

The intravenous tubing set of the fourth aspect may include one or more of the previous embodiments and, optionally, wherein the first line of tubing is in fluid communication with a coupler, wherein the coupler is able to engage the vented spike.

The intravenous tubing set of the fourth aspect may include one or more of the previous embodiments and, optionally, wherein the coupler able to engage the vented spike includes a coupling mechanism comprising a spinlock or a Luer lock.

The intravenous tubing set of the fourth aspect may include one or more of the previous embodiments and, optionally, wherein the coupler able to engage the vented spike includes a cap operable to cover a distal end of the coupler.

The intravenous tubing set of the fourth aspect may include one or more of the previous embodiments and, optionally, a third line of tubing in fluid communication with a second end of the dual port chamber and in fluid communication with a coupler, wherein the coupler is able to engage an instrument attached to a patient at the intravenous site.

The intravenous tubing set of the fourth aspect may include one or more of the previous embodiments and, optionally, a clamp operable to control a fluid flow through the third line of tubing.

The intravenous tubing set of the fourth aspect may include one or more of the previous embodiments and, optionally, wherein the coupler able to engage the instrument includes a coupling mechanism comprising a spinlock or a Luer lock.

The intravenous tubing set of the fourth aspect may include one or more of the previous embodiments and, optionally, wherein the coupler able to engage the instrument includes a cap operable to cover a distal end of the coupler.

The intravenous tubing set of the fourth aspect may include one or more of the previous embodiments and, optionally, an additional connector separating the third line of tubing into a first section and a second section. The first section of the third line of tubing is in fluid communication with the second end of the dual port chamber and in fluid communication with a first end of the additional connector. The second section of the third line of tubing is in fluid communication with a second end of the additional connector and in fluid communication with the additional connector.

The intravenous tubing set of the fourth aspect may include one or more of the previous embodiments and, optionally, wherein a first end of the additional connector includes a first branch and a second branch, and wherein the first section of the third line of tubing is in fluid communication with the first branch of the additional connector.

The intravenous tubing set of the fourth aspect may include one or more of the previous embodiments and, optionally, wherein the second branch of the additional connector is operable to provide access to the second section of the third line of tubing.

The intravenous tubing set of the fourth aspect may include one or more of the previous embodiments and, optionally, wherein the second end of the additional connector and the second branch of the first end of the additional connector are substantially collinear along a longitudinal axis through the additional connector, and wherein the first branch of the first end of the additional connector is set at an angle to the longitudinal axis through the additional connector.

The intravenous tubing set of the fourth aspect may include one or more of the previous embodiments and, optionally, wherein the dual port chamber includes a cap at the first end.

The intravenous tubing set of the fourth aspect may include one or more of the previous embodiments and, optionally, wherein the first line of tubing and the second line of tubing are separately in fluid communication with the cap.

The intravenous tubing set of the fourth aspect may include one or more of the previous embodiments and, optionally, wherein the dual port chamber includes a port at a second end opposite to the first end of the dual port chamber.

These and other advantages will be apparent from the disclosure contained herein. The above-described embodiments, objectives, and configurations are neither complete nor exhaustive. The Summary is neither intended nor should it be construed as being representative of the full extent and scope of the present disclosure. Moreover, references made herein to “the present disclosure,” or aspects thereof should be understood to mean certain embodiments of the present disclosure and should not necessarily be construed as limiting all embodiments to a particular description. The present disclosure is set forth in various levels of detail in the Summary as well as in the attached drawings and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the drawings.

### BRIEF DESCRIPTION OF THE DRAWINGS

Those of skill in the art will recognize that the following description is merely illustrative of the principles of the disclosure, which may be applied in various ways to provide many different alternative embodiments. This description is made for illustrating the general principles of the teachings of this disclosure and is not meant to limit the concepts disclosed herein.

The accompanying drawings, which are incorporated in and constitute a part of the specification, illustrate embodiments and together with the general description of the disclosure given above and the detailed description of the drawings given below, serve to explain the principles of the disclosure.

FIG. 1A illustrates an intravenous tubing set, in accordance with one or more embodiments of the present disclosure;

FIG. 1B illustrates a dual port chamber of the intravenous tubing set of FIG. 1A;

FIG. 1C illustrates the intravenous tubing set of FIG. 1A;

FIG. 1D illustrates a portion of the intravenous tubing set of FIG. 1C in fluid communication with a first container including a pharmaceutical composition and a second container including a solution;

FIG. 1E illustrates another embodiment of the intravenous tubing set of FIG. 1A;

FIG. 1F illustrates another embodiment of the intravenous tubing set of FIG. 1A;

FIG. 2 illustrates a first variation of the intravenous tubing set of FIG. 1A, in accordance with one or more embodiments of the present disclosure;

FIG. 3 illustrates a second variation of the intravenous tubing set of FIG. 1A, in accordance with one or more embodiments of the present disclosure;

FIG. 4 illustrates a third variation of the intravenous tubing set of FIG. 1A, in accordance with one or more embodiments of the present disclosure;

FIG. 5 illustrates a fourth variation of the intravenous tubing set of FIG. 1A, in accordance with one or more embodiments of the present disclosure;

FIG. 6A illustrates a fifth variation of the intravenous tubing set of FIG. 1A, in accordance with one or more embodiments of the present disclosure;

FIG. 6B illustrates a first portion of the intravenous tubing set of FIG. 6A in fluid communication with a first container including a pharmaceutical composition and a second container including a solution;

FIG. 6C illustrates a second portion of the intravenous tubing set of FIG. 6B in fluid communication with the first container including the pharmaceutical composition and the second container including the solution;

FIG. 6D illustrates the intravenous tubing set of FIG. 6A; and

FIG. 7 illustrates a flow diagram of a method or process for the operation and use of the intravenous tubing sets as described throughout the present disclosure.

It should be understood that the drawings are not necessarily to scale, and various dimensions may be altered. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Although the following text sets forth a detailed description of numerous different embodiments, it should be understood that the legal scope of the description is defined by the words of the claims set forth at the end of this disclosure. The Detailed Description is to be construed as exemplary only and does not describe every possible embodiment since describing every possible embodiment of the intravenous tubing set would be impractical, if not impossible. Numerous alternative embodiments could be implemented, using either current technology or technology developed after the filing date of this patent, which would still fall within the scope of the claims. Additionally, any combination of features shown in the various figures can be used to create additional embodiments of the present disclosure. Thus, dimensions, aspects, and features of one embodiment of the intravenous tubing set can be combined with dimensions, aspects, and features of another embodiment of the intravenous tubing set to create the claimed embodiment.

In general, embodiments of the present disclosure are directed to a system and method for administering a pharmaceutical composition with an intravenous tubing set. The intravenous tubing set includes a first spike on a first line for a pharmaceutical composition and a second spike on a second line for a solution. The first line and the second line are in fluid communication with a dual port chamber, which is in fluid communication with a coupler that is able to engage an instrument attached to a patient at an intravenous site. In some embodiments, the pharmaceutical composition is administered using a gravity feed via the intravenous tubing set. In some embodiments, the first spike is a vented spike and the second spike is a non-vented spike.

Embodiments of the present disclosure are also directed to an intravenous tubing set capable of administering a dose of pharmaceutical composition that is separated into different infusion vials. The intravenous tubing set should include a vented spike that is insertable into the different infusion vials. The vented spike should not need to be changed between infusion vials due to clogging of a filter within the vented spike, or the filter being made wet, by the pharmaceutical composition from the first infusion vial. The vented spike should be insertable into the stopper of the infusion vial without unseating or dislodging the stopper, which may cause leakage from the infusion vial (i.e., due to the suspension of the infusion vial for purposes of gravity feeding through the intravenous tubing set).

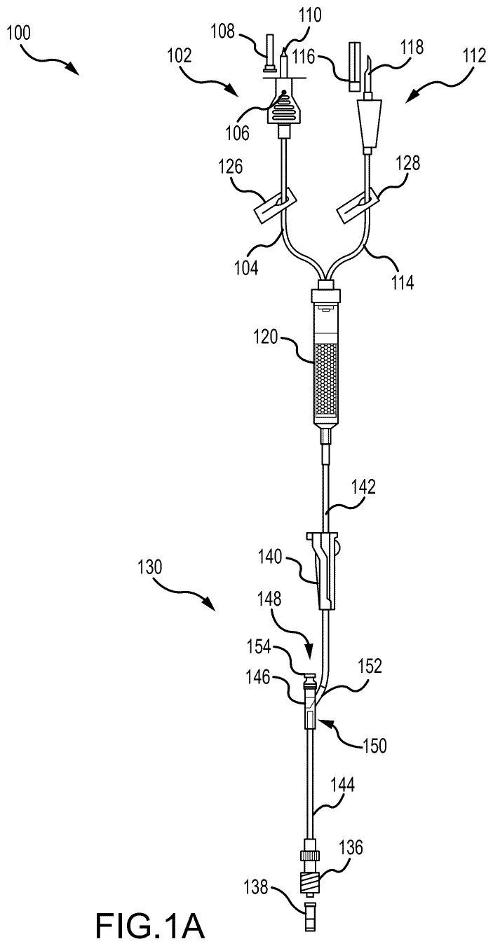

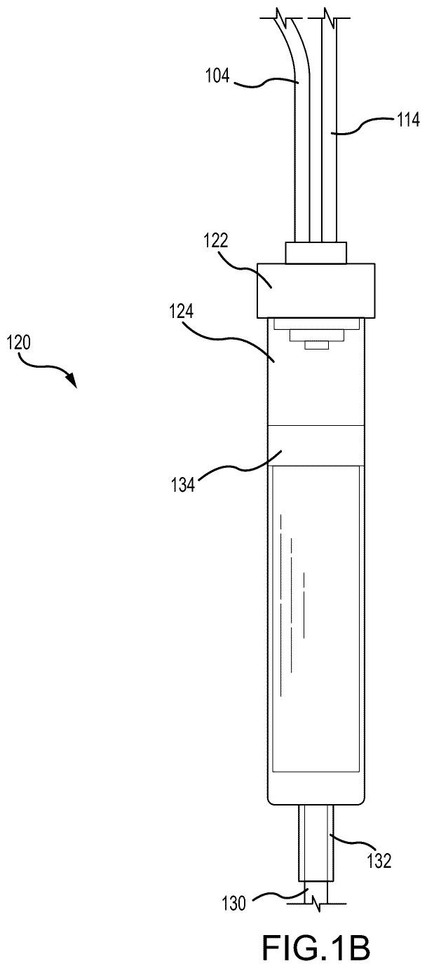

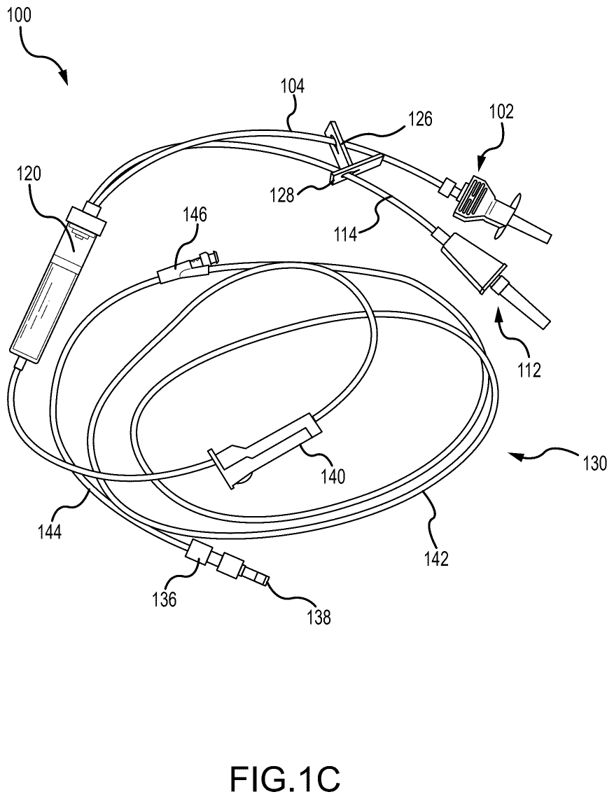

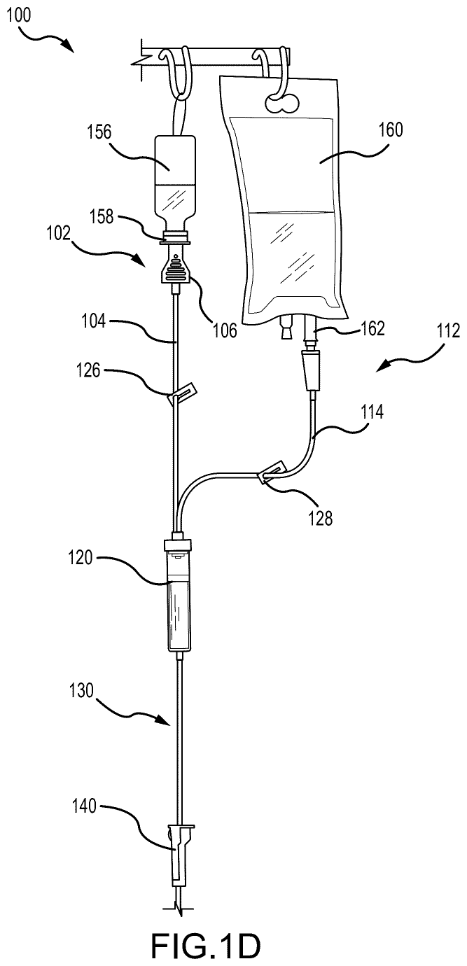

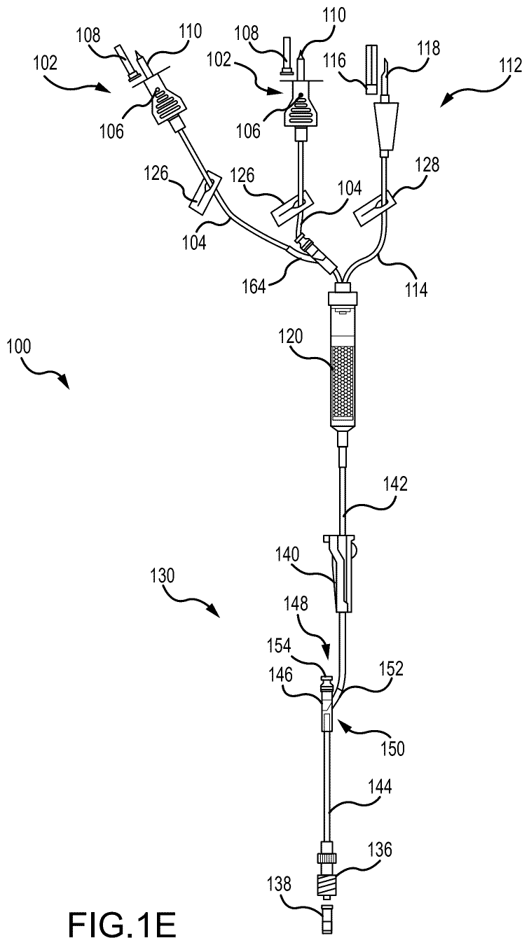

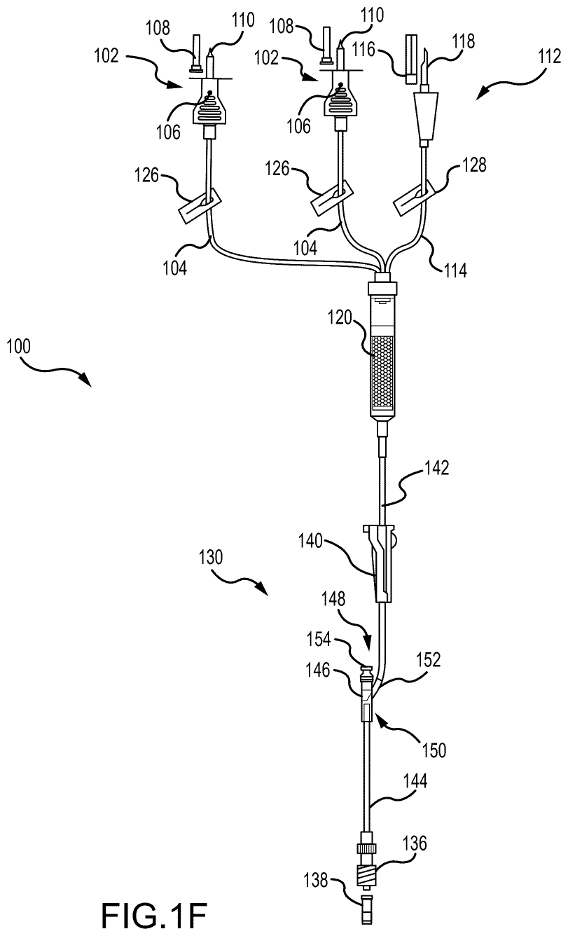

FIGS. 1A-IF in general illustrate an intravenous tubing set 100, in accordance with one or more embodiments of the present disclosure. It is noted that “intravenous tubing” and “IV tubing” (along with other variants of “intravenous” versus “IV”) may be considered equivalent, for purposes of the present disclosure.

The intravenous tubing set 100 includes a spike 102 in fluid communication with a line of tubing 104. The spike 102 is insertable into a container 156 operable to store a pharmaceutical composition. For example, the spike 102 may be a vented spike operable to allow airflow into the container 156 to allow for air to enter into the container 156 while a pharmaceutical composition stored within the container 156 flows through the line of tubing 104. For instance, this may be beneficial where the container 156 includes rigid or non-deformable sidewalls (i.e., such as a glass or rigid plastic infusion vial). In this example, the spike 102 may include a filter 106 for the airflow into the spike 102.

It is noted herein that the pharmaceutical composition as described throughout the present disclosure may include, but is not limited to, a 100 milliliter (mL) dose comprising Natural Killer (NK) cellular products (e.g., including, but not limited to, PD-L1 t-haNK or CD19 t-haNK), where the 100 mL dose is separated into two 50 mL infusion vials 156. In general, the pharmaceutical composition may be any investigational product or other solution including a drug substance that can be supplied via gravity feed from a container 156 (e.g., that may be rigid, such as an infusion vial). In embodiments, the spike 102 includes a cap 108 to assist in ensuring the tip 110 of the spike 102 remains sterile or otherwise uncontaminated (i.e., unclogged and/or not wet) prior to insertion into the container 156.

The intravenous tubing set 100 includes a spike 112 in fluid communication with a line of tubing 114. For example, the spike 112 may be a non-vented spike. The spike 112 is insertable into a container 160 (e.g., an IV bag, an infusion vial, or the like) operable to store a solution. For example, the solution may include, but is not limited to, a saline solution or other solution that (a) primes or flushes the intravenous tubing set, (b) combines with the pharmaceutical composition in the container 156, or (c) is an additional pharmaceutical composition. In embodiments, the spike 112 includes a cap 116 to assist in promoting a sterile spike tip 118 prior to insertion into the container 160.

In some configurations of the intravenous tubing set 100, the spike 102 is shorter than the spike 112. For example, the spike 102 may have an inner diameter of 0.162 inches , an outer diameter of 0.303 inches, and a length of 2.36 inches. By way of another example, the spike 112 may have an inner diameter of 0.162 inches, an outer diameter of 0.232 inches, and a length of 3.27 inches. It is noted herein that the longer spike 112 may be used to supply the solution from the container 160 to prime the intravenous tubing set 100 (i.e., with solution from the container 160) prior to administering the pharmaceutical composition from the container 156 via the spike 102. In addition, it is noted herein that the longer spike 112 may be used to supply the solution from the container 160 to flush the intravenous tubing set 100 (i.e., with solution from the container 160) after administering the pharmaceutical composition from the container 156 via the spike 102.

In embodiments, the line of tubing 104 and the line of tubing 114 are each in fluid communication with a dual port chamber 120. For example, as illustrated in FIG. 1B, the dual port chamber 120 may include a cap 122 at a first end of the dual port chamber 120, where the cap 122 includes one or more openings into which the line of tubing 104 and the line of tubing 114 are inserted. By way of another example, the dual port chamber 120 may include one or more ports to which the line of tubing 104 and/or the line of tubing 114 are coupled (e.g., either internally with the line of tubing being inserted into the port, or externally with the port being inserted into the line of tubing). It is noted herein that the line of tubing 104 and the line of tubing 114 may be in fluid communication with the same opening or with the same port of the cap 122, or alternatively may be in fluid communication with different openings or ports of the cap 122, without departing from the scope of the present disclosure. Further, it is contemplated that the cap 122 in able to receive (or includes) a connector to which the line of tubing 104 and the line of tubing 114 each couple such that the line of tubing 104 is coupled to a first branch of the connector, the line of tubing 114 is coupled to a second branch of the connector, and a third branch of the connector is coupled to the cap 122. The dual port chamber 120 additionally includes a housing 124 to which the cap 122 is coupled.

Fluid flow (including the rate thereof) through the line of tubing 104 from the spike 102 to the dual port chamber 120 may be controlled via a clamp 126. For example, the clamp 126 may be a slide clamp usable to start or stop the fluid flow through the line of tubing 104. By way of another example, the clamp 126 may be a roller clamp usable to control the rate of fluid flow through the line of tubing 104. It is noted herein that reducing the flow rate to 0 (or allowing the flow rate to be greater than 0) via the roller clamp effectively results in a stopping or starting of the fluid flow within the line of tubing 104.

Fluid flow (including the rate thereof) through the line of tubing 114 from the spike 112 to the dual port chamber 120 may be controlled via a clamp 128. For example, the clamp 128 may be a slide clamp usable to start or stop the fluid flow through the line of tubing 114. By way of another example, the clamp 128 may be a roller clamp usable to control the rate of fluid flow through the line of tubing 114. It is noted herein that reducing the flow rate to 0 (or allowing the flow rate to be greater than 0) via the roller clamp effectively results in a stopping or starting of the fluid flow within the line of tubing 114.

In embodiments, a line of tubing 130 is in fluid communication with a port 132 at a second end of the dual port chamber 120. For example, the line of tubing 130 may be internally coupled to the port 132, with the line of tubing 130 being inserted into the port 132. By way of another example, the line of tubing 130 may be externally coupled to the port 132, with the port 132 being inserted into the line of tubing 130. Fluid that passes through the housing 124 may exit through the port 132 and into the line of tubing 130.

It should be understood that the cap 122 may include one set of openings or ports for the dual port chamber 120, and the housing 124 may include a second set of openings or ports (i.e., the port 132) for the dual port chamber 120, for purposes of the present disclosure. However, it is contemplated that the dual port chamber 120 may be a sealed unit with openings and/or ports at both a first end and a second opposite end of the housing 124, such that no cap 122 is necessary, without departing from the scope of the present disclosure.

In some non-limiting embodiments, the housing 124 may optionally include a filter 134, such that the dual port chamber 120 is a filter chamber. For example, the pharmaceutical composition supplied by the line of tubing 104 from the container 156 and/or the solution supplied by the line of tubing 114 from the container 160 to the dual port chamber 120 may pass through the filter 134 prior to the fluid exiting the dual port chamber 120 via the port 132 and the line of tubing 130. It is noted that the filter 134 may include a blood filter that ranges from 170 microns to 260 micron. For example, the filter 134 may be a 200-micron blood filter.

It is noted herein that one or more of the line of tubing 104, the line of tubing 114, the line of tubing 130, and the dual port chamber 120 may be clear or translucent to allow for visualization of the pharmaceutical composition and/or the solution through the intravenous tubing set 100. However, it should be understood that one or more of the line of tubing 104, the line of tubing 114, the line of tubing 130, and the dual port chamber 120 may be partially or substantially opaque, without departing from the scope of the present disclosure.

In embodiments, the line of tubing 130 is in fluid communication with a coupler 136. For example, the coupler 136 is able to engage an instrument (i.e., a needle or other port) attached to a patient at an intravenous site.

In some embodiments, the coupler 136 includes a coupling mechanism able to engage the instrument attached to the patient at the intravenous site. For example, the coupling mechanism may include, but is not limited to, a set of interlocking components such as complementary threading, a spinlock or a Luer lock, or the like. It is noted that the coupling mechanism should be scaled and/or leak-proof. Additionally, it is noted that the coupling mechanism may be a one-way coupling mechanism to prevent disengagement of the coupler 136 from the instrument attached at the intravenous site. It is noted herein, however, that the line of tubing may be directly coupled to the coupler 136, either internally with the line of tubing 130 being inserted into the coupler 136, or externally with the coupler 136 being inserted into the line of tubing 130, without departing from the scope of the present disclosure.

In some embodiments, the coupler 136 includes a cap 138 that assists in maintaining sterility of the distal end of the coupler 136 prior to being coupled to the instrument attached to the patient at the intravenous site. For example, the cap 138 may be a breather.

Fluid flow (including the rate thereof) through the line of tubing 130 to the coupler 136 from the dual port chamber 120 may be controlled via a clamp 140. For example, the clamp 140 may be a roller clamp usable to control the rate of fluid flow through the line of tubing 130. It is noted herein that reducing the flow rate to 0 (or allowing the flow rate to be greater than 0) via the roller clamp effectively results in a stopping or starting of the fluid flow within the line of tubing 130. By way of another example, the clamp 140 may be a slide clamp usable to start or stop the fluid flow through the line of tubing 130.

In some embodiments, the dual port chamber 120 is in direct fluid communication with the coupler 136 via the line of tubing 130. In other embodiments, the line of tubing 130 is separated into multiple sections coupled together via at least one connector.

For example, the line of tubing 130 may be separated into a first section of tubing 142 and a second section of tubing 144. The first section of tubing 142 and the second section of tubing 144 may be in fluid communication with a connector 146. For instance, the first section of tubing 142 of the third line of tubing 130 may be in fluid communication with the second end of the dual port chamber 120, and in fluid communication with a first end 148 of the connector 146. In addition, the second section of tubing 144 of the third line of tubing 130 may be in fluid communication with a second end 150 of the connector 146 and in fluid communication with the coupler 136. In some embodiments, the connector 146 is a needleless connector.

It is noted herein that the connector 146 may include one or more branches on the first end 148 and/or the second end 150. In one non-limiting example, the connector 146 may include a first branch 152 and a second branch 154 on the first end 148. In this non-limiting example, the connector 146 may be a Y-connector or a T-connector. Where the connector 146 is a Y-connector or a T-connector, the second branch 154 may be aligned with the second end 150 by being coaxial or sharing a longitudinal axis through the connector 146, such that the connector 146 provides access for a medical professional (i.e., a technician, nurse, doctor, or the like) to the second section of tubing 144 of the line of tubing 130, while the first branch 152 is set an angle relative to the longitudinal axis. However, it is noted herein that both the first branch 152 and the second branch 154 may be set at an angle relative to the longitudinal axis through the second end 150 of the connector 146, and that the respective angles may be substantially the same or may be different, without departing from the scope of the present disclosure.

It is noted herein that the clamp 140 may be positioned on either the first section of tubing 142 or the second section of tubing 144 (or, in general, any section of the line of tubing 130), without departing from the scope of the present disclosure.

As illustrated in FIG. 1D, in some embodiments the spike 102 is usable to administer a dose of a pharmaceutical composition, where the dose is separated into one or more containers. Where the container 156 is an infusion vial, the spike 102 is insertable into a stopper 158 of the container 156. In addition, where the container 160 is an IV bag, the spike 112 is insertable into a port 162 of the container 160.

In one non-limiting example, the dose of the pharmaceutical composition may be in a single container 156. In another non-limiting example, the dose of the pharmaceutical composition may be in multiple containers 156 (e.g., due to size constraints, timing between administration of portions of the dose, or for other reasons specific to the pharmaceutical composition). It is contemplated that the same spike 102 may be couplable to the multiple containers 156 in succession during administration. For example, a medical professional may remove a first container 156 and sterilize the spike 102 (i.e., by performing hand hygiene, putting on clean gloves, and applying alcohol to at least the tip 110 of the spike 102) immediately prior to inserting the tip 110 into the second container 156 using an aseptic non-touch technique (i.e., so that the stopper 158 of the container 156 is not touched once the tip 110 is cleaned). In this regard, administration of each portion of the dose of the pharmaceutical composition may be controlled by the medical professional.

Although FIG. 1A illustrates a single line of tubing 104 and spike 102, FIG. 1E illustrates that the single line of tubing 104 may be further branched to include multiple spikes 102. For example, the line of tubing 104 may include first portion in fluid communication with the dual port chamber 120 and a connector. The connector is in fluid communication via a connector 164 with two branch lines that each include a spike 102. Each branch line with respective spike 102 is for a container 156 including a portion of the dose of the pharmaceutical composition. It is noted that each branch line of the line of tubing 104 may additionally or alternatively include a clamp 126. In this regard, the multiple portions of the pharmaceutical composition may be coupled to the intravenous tubing set at any point during administration. In addition, with the inclusion of the clamps 126 on each of the branch lines, administration of each portion of the dose of the pharmaceutical composition may be controlled by a medical professional.

In addition, although FIG. 1A illustrates a single line of tubing 104 and spike 102, Fig. IF illustrates that multiple separate sub-assemblies for the administration of the pharmaceutical composition that each include a line of tubing 104 and a spike 102 may instead be in fluid communication with the dual port chamber 120. For example, the intravenous tubing set 100 may include two sub-assemblies that each include the line of tubing 104 and the spike 102. Each sub-assembly is for a container 156 including a portion of the dose of the pharmaceutical composition. It is noted that each branch line of the line of tubing 104 may additionally include a clamp 126. In this regard, the multiple portions of the pharmaceutical composition may be coupled to the intravenous tubing set at any point during administration. In addition, with the inclusion of the clamps 126 on each of the sub-assemblies, administration of each portion of the dose of the pharmaceutical composition may be controlled by a medical professional.

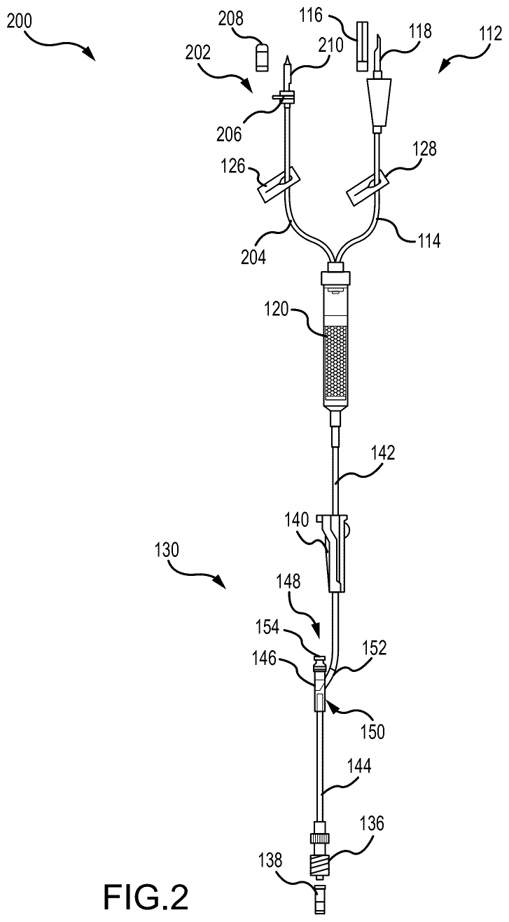

FIG. 2 illustrates a variation of the intravenous tubing set 100 of FIG. 1A, or intravenous tubing set 200, in accordance with one or more embodiments of the present disclosure. It should be understood that aspects and/or components of the intravenous tubing set 100 may be shared with and/or combinable with one or more aspects and/or components of the intravenous tubing set 200 without departing from the scope of the present disclosure, unless otherwise noted. In particular, it should be understood that the intravenous tubing set 200 may include alternative builds like those illustrated in FIGS. 1E and IF for the intravenous tubing set 100, without departing from the scope of the present disclosure.

As illustrated in FIG. 2, the intravenous tubing set 200 includes a spike 202 coupled to tubing 204 and with a filter 206. The spike 202 includes a cap 208 and a sterile tip 210 that is shorter and smaller in diameter than the spike 102 of FIG. 1A. For example, the spike 202 may be a vented spike. It was determined through testing, however, that the sterile tip 210 was too short and/or too small in diameter, causing it to be easily pulled out or dislodged from a stopper 158 of the infusion vial 156 and creating the potential for leakage from the stopper 158 (i.e., from the hole made into the stopper 158 by the sterile tip 210). In some instances, the leakage from the stopper 158 clogged the filter 206 and/or made the filter 206 wet. As such, the length and diameter of the spike 102 as illustrated in FIG. 1A was determined to be more beneficial for the operation of the intravenous tubing set 100.

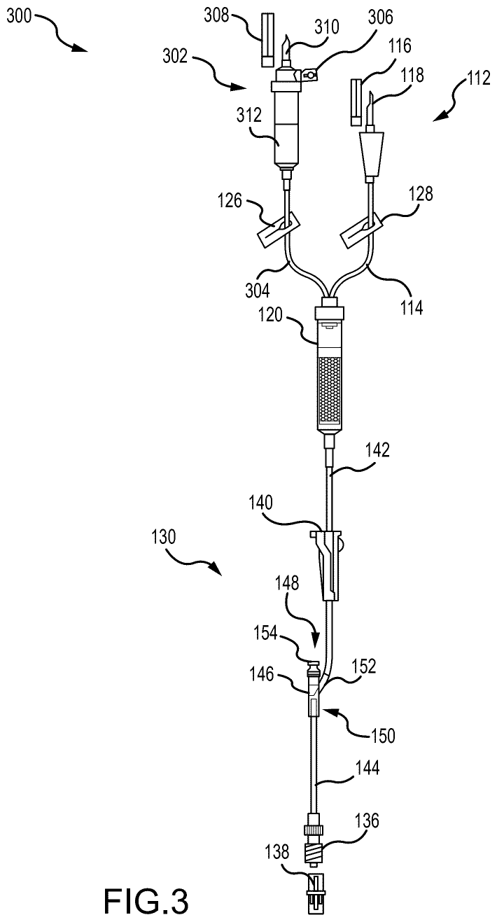

FIG. 3 illustrates a variation of the intravenous tubing set 100 of FIG. 1A, or intravenous tubing set 300, in accordance with one or more embodiments of the present disclosure. It should be understood that aspects and/or components of the intravenous tubing set 300 may be shared with and/or combinable with one or more aspects and/or components of the intravenous tubing set 100 and/or 200 without departing from the scope of the present disclosure, unless otherwise noted. In particular, it should be understood that the intravenous tubing set 300 may include alternative builds like those illustrated in FIGS. 1E and IF for the intravenous tubing set 100, without departing from the scope of the present disclosure.

As illustrated in FIG. 3, the intravenous tubing set 300 includes a spike 302 coupled to tubing 304 and with a filter 306. The spike 302 includes a cap 308 and a sterile tip 310 that is shorter and larger in diameter than the spike 102 of FIG. 1A. For example, the spike 302 may be a vented spike. The spike 302 includes a chamber 312.

It was determined through testing, however, that the sterile tip 310 was too short and/or too large in diameter to effectively push through a stopper 158 of the infusion vial 156, instead causing the stopper 158 to be pushed into the infusion vial 156 and creating the potential for leakage from the spike 302. In addition, improper contamination of the filter 306 may occur. Further, the chamber 312 was determined to not be a necessary part of the vented spike 302. As such, the length and diameter of the spike 102 as illustrated in FIG. 1A was determined to be more beneficial for the operation of the intravenous tubing set 100.

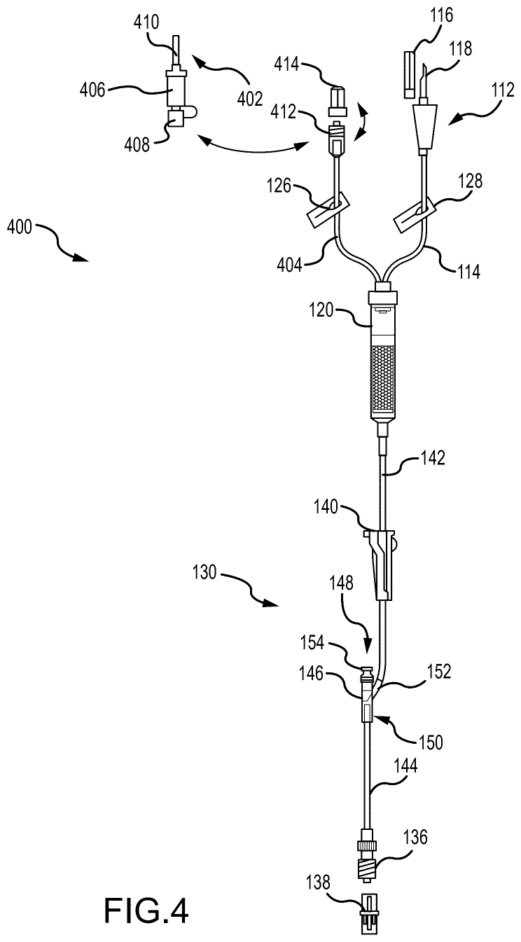

FIG. 4 illustrates a variation of the intravenous tubing set 100 of FIG. 1A, or intravenous tubing set 400, in accordance with one or more embodiments of the present disclosure. It should be understood that aspects and/or components of the intravenous tubing set 400 may be shared with and/or combinable with one or more aspects and/or components of the intravenous tubing set 100, 200, and/or 300 without departing from the scope of the present disclosure, unless otherwise noted. In particular, it should be understood that the intravenous tubing set 400 may include alternative builds like those illustrated in FIGS. 1E and IF for the intravenous tubing set 100, without departing from the scope of the present disclosure.

As illustrated in FIG. 4, the intravenous tubing set 400 includes a spike 402 coupled to tubing 404 and with a filter 406. The spike 402 includes a cap 408 and a sterile tip 410 that is shorter and smaller in diameter than the spike 102 of FIG. 1A. For example, the spike 402 may be a non-vented spike.

In embodiments, the spike 402 is separate from the intravenous tubing set 400, and is couplable to a coupler 412 on the line of tubing 404. In some embodiments, the spike 402 is coupled to the coupler 412 via a coupling mechanism able to receive and connect with the spike 402. For example, the coupling mechanism may include, but is not limited to, a set of interlocking components such as complementary threading, a spinlock or a Luer lock, or the like. It is noted that the coupling mechanism may be sealed and/or leak-proof. Additionally, it is noted that the coupling mechanism may be a one-way coupling to prevent disengagement of the spike 402 from the line of tubing 604.

In some embodiments, the coupler 412 may include a cap 414 to assist in maintaining sterility of the coupler 412 (e.g., when the spike 402 is not attached to the coupler 412).

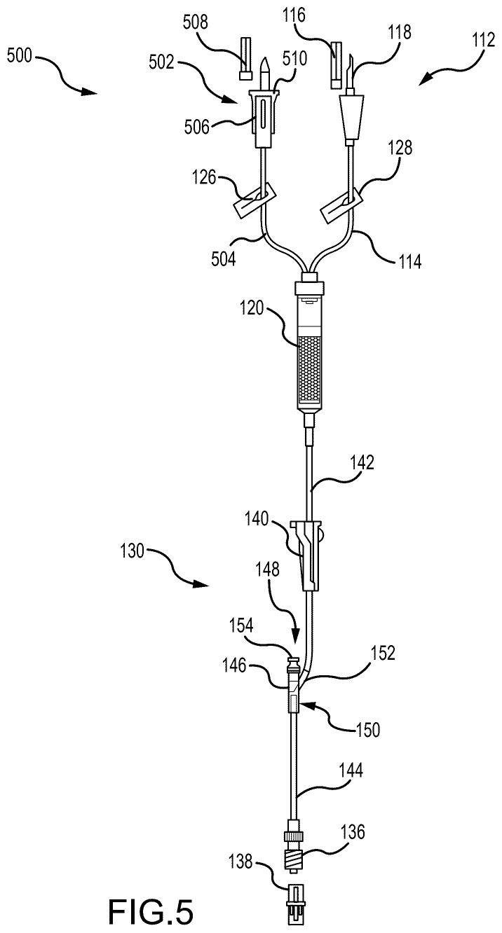

FIG. 5 illustrates a variation of the intravenous tubing set 100 of FIG. 1A, or intravenous tubing set 500, in accordance with one or more embodiments of the present disclosure. It should be understood that aspects and/or components of the intravenous tubing set 500 may be shared with and/or combinable with one or more aspects and/or components of the intravenous tubing set 100, 200, 300, and/or 400 without departing from the scope of the present disclosure, unless otherwise noted. In particular, it should be understood that the intravenous tubing set 500 may include alternative builds like those illustrated in FIGS. 1E and IF for the intravenous tubing set 100, without departing from the scope of the present disclosure.

As illustrated in FIG. 5, the intravenous tubing set 500 includes a spike 502 coupled to tubing 504 and with a filter 506. The spike 502 includes a cap 508 and a sterile tip 510 that is shorter and smaller in diameter than the spike 102 of FIG. 1A. For example, the spike 502 may be a vented spike. It was determined through testing, however, that the tip 510 provided difficult to sterilize. As such, the length and diameter of the spike 102 as illustrated in FIG. 1A was determined to be more beneficial for the operation of the intravenous tubing set 100.

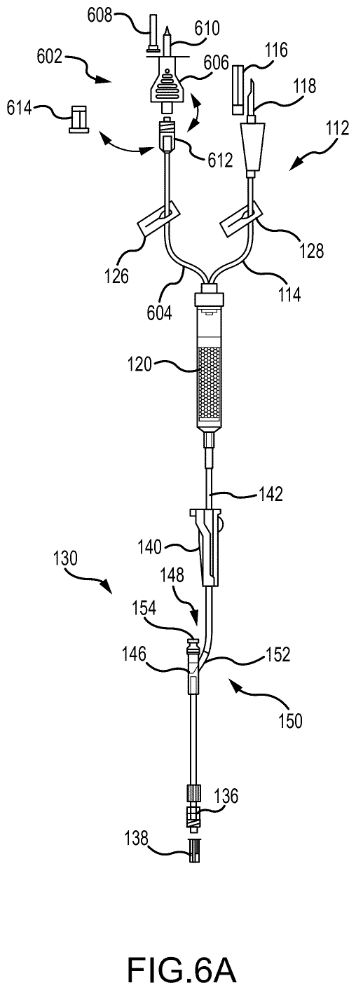

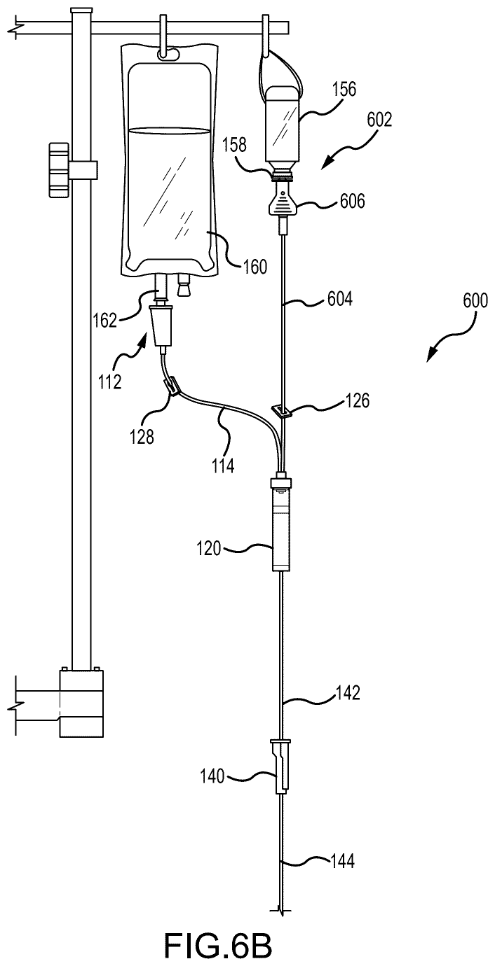

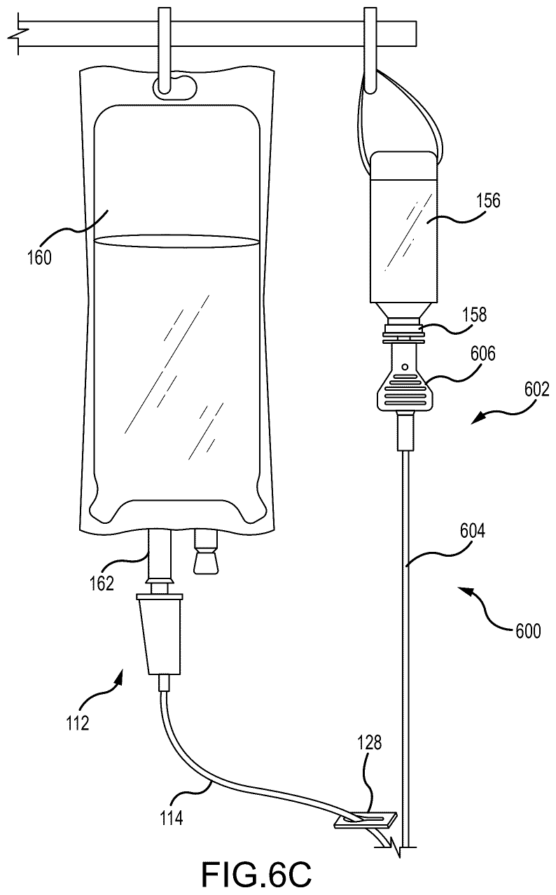

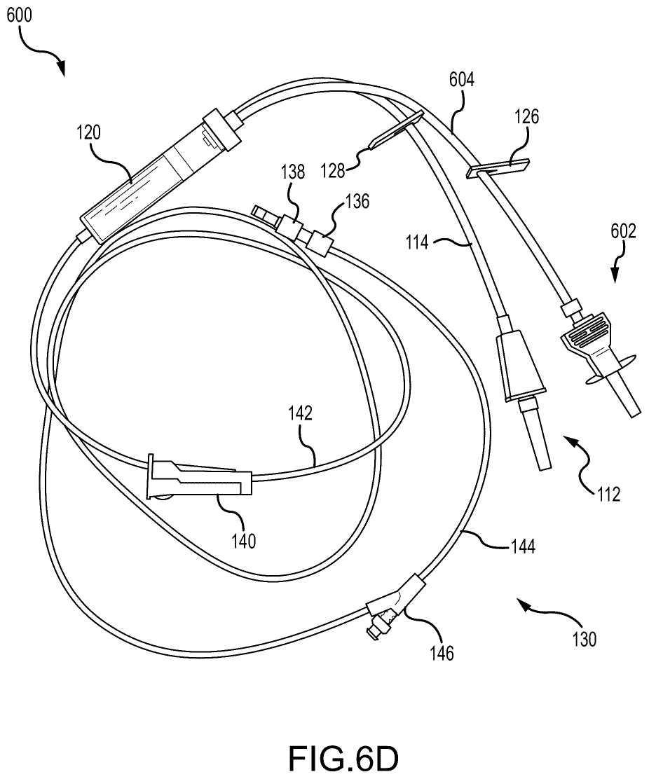

FIG. 6A-6D in general illustrate a variation of the intravenous tubing set 100 of FIG. 1A, or intravenous tubing set 600, in accordance with one or more embodiments of the present disclosure. It should be understood that aspects and/or components of the intravenous tubing set 600 may be shared with and/or combinable with one or more aspects and/or components of the intravenous tubing set 100, 200, 300, 400, and/or 500 without departing from the scope of the present disclosure, unless otherwise noted. In particular, it should be understood that the intravenous tubing set 600 may include alternative builds like those illustrated in FIGS. 1E and IF for the intravenous tubing set 100, without departing from the scope of the present disclosure.

As illustrated in FIGS. 6A-6D, the intravenous tubing set 600 includes a spike 602 coupled to tubing 604 and with a filter 606. The spike 602 includes a cap 608 and a sterile tip 610. For example, the spike 602 may be a vented spike.

It was determined through testing that the spike 102 (e.g., when vented) may only operate when subjected to pressure up to a pre-determined threshold (e.g., up to approximately 2.9 pounds per square inch (PSI)). In contrast, the spike 602 (e.g., when vented) may be able to handle an increased amount of pressure as compared to the spike 102. This ability to handle an increased amount of pressure by the spike 602 was determined to potentially be more beneficial for the operation of the intravenous tubing set 600, as compared to the spike 102 of the intravenous tubing set 100.

In embodiments, the spike 602 is separate from the intravenous tubing set 600, and is couplable to a coupler 612 on the line of tubing 104. In some embodiments, the spike 602 is coupled to the coupler 612 via a coupling mechanism able to receive and connect with the spike 602. For example, the coupling mechanism may include, but is not limited to, a set of interlocking components such as complementary threading, a spinlock or a Luer lock, or the like. It is noted that the coupling mechanism may be sealed and/or leak-proof. Additionally, it is noted that the coupling mechanism may be a one-way coupling to prevent disengagement of the spike 602 from the line of tubing 604.

In some embodiments, the coupler 612 may include a cap 614 to assist in maintaining sterility of the coupler 612 (e.g., when the spike 602 is not attached to the coupler 612).

Thus, as illustrated by a comparison of the intravenous tubing set builds illustrated in FIGS. 1A-6D, testing and experimentation was necessary to determine the appropriate combination of spikes. From that testing and experimentation, it was determined a vented spike that would not become wet or clogged and thus be reusable with multiple infusion vials, and that would not unseat or dislodge the stopper in the infusion vial, was a beneficial configuration for the intravenous tubing set of the present application as compared to known intravenous tubing sets. As the vented spike is reusable, the intravenous tubing set of the present application is more cost-effective than previously known solutions (i.e., such as single-use intravenous tubing sets). In addition, as the vented spike may be interchangeable with another vented spike, the intravenous tubing set of the present application is more cost-effective than previously known solutions by only needing to interchange the individual vented spike (i.e., as compared to single-use full-build intravenous tubing sets).

In some embodiments, the respective line of tubing 104, 204, 304, 404, 504, 604 of the intravenous tubing sets 100, 200, 300, 400, 500, 600 may be approximately 10 inches in length. In addition, the line of tubing 114 may be approximately 10 inches in length. Further, the first section of tubing 142 of the line of tubing 130 may be approximately 60 inches in length, and the second section of tubing 144 of tubing of the line of tubing 130 may be approximately 10 inches in length. As such, the intravenous tubing sets 100, 200, 300, 400, 500, 600 may each have a total length of approximately 80 inches of tubing (e.g., with the lines of tubing 114 and 130 with sections 142 and 144 being in series, and the line of tubing 114 and respective line of tubing 104, 204, 304, 404, 504, 604 being in parallel, as illustrated in the various Figs.). However, it should be understood that the lengths of the tubing 104, 204, 304, 404, 504, 604, 114, and 130 with sections 142 and 144, and/or the total length of tubing of the intravenous tubing sets 100, 200, 300, 400, 500, 600, are not limited to the above example. Rather, the respective lengths of the tubing 104, 204, 304, 404, 504, 604, 114, and 130 with sections 142 and 144, and/or the total length of tubing of the intravenous tubing sets 100, 200, 300, 400, 500, 600, may be shorter or longer than in the above example, without departing from the scope of the present disclosure.

In one non-limiting example, the tubing 104, 204, 304, 404, 504, 604, 114, and 130 with sections 142 and 144 of the intravenous tubing sets 100, 200, 300, 400, 500, 600, respectively, has an inner diameter that is approximately 0.110 inches (2.79 millimeters), and an outer diameter that is approximately 0.162 inches (4.11 millimeters). It is contemplated, however, that at least some of the tubing 104, 204, 304, 404, 504, 604, 114, and 130 with sections 142 and 144 of the intravenous tubing sets 100, 200, 300, 400, 500, 600, respectively, may have a different inner diameter and/or outer diameter.

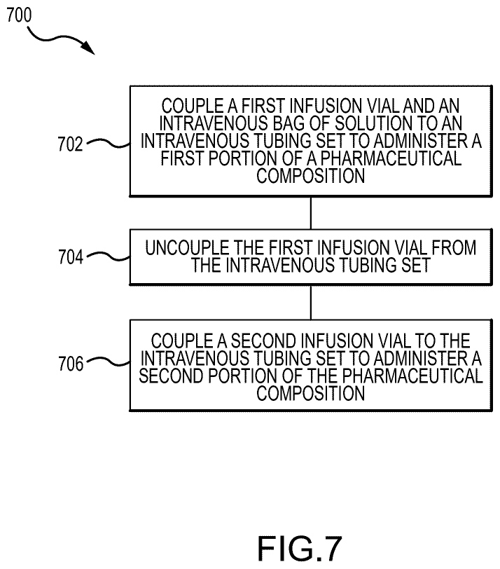

FIG. 7 is a method or process flow diagram 700 illustrating the administration of a pharmaceutical composition using the intravenous tubing sets 100, 200, 300, 400, 500, 600 in accordance with one or more embodiments or the present disclosure. While a general order for the steps of the method or process is shown in FIG. 7, the method or process can include more or fewer steps or can arrange the order of the steps differently (including simultaneously, substantially simultaneously, or sequentially) than those shown in FIG. 7. It is noted that the method or process of FIG. 7 shall be explained with reference to the components, devices, subassemblies, environments, etc. described in conjunction with FIGS. 1A-6D. For example, it is noted that the embodiments as illustrated in FIGS. 1A-6D should be understood as reading on the embodiments described with respect to FIG. 7, and vice versa, without departing from the scope of the present disclosure.

In embodiments, a first infusion vial and an intravenous bag of solution are coupled 702 to an intravenous tubing set to administer a first portion of a pharmaceutical composition. For example, a first infusion vial 156 may be coupled to the spike 102, 202, 302, 402, 502, 602 and the intravenous bag of solution may be coupled to the spike 112 of the intravenous tubing sets 100, 200, 300, 400, 500, 600, respectively as described throughout the present disclosure.

In embodiments, the first infusion vial is uncoupled 704 from the intravenous tubing set. For example, the first infusion vial 156 may be uncoupled or removed from the spike 102, 202, 302, 402, 502, 602.

In embodiments, a second infusion vial is coupled 706 to the intravenous tubing set to administer a second portion of the pharmaceutical composition. For example, the first infusion vial 156 may be uncoupled or removed from the spike 102, 202, 302, 402, 502, 602 prior to the coupling of a second infusion vial 156 to the spike 102, 202, 302, 402, 502, 602 and administration of the contents of the second infusion vial 156. By way of another example, the second infusion vial 156 may be coupled to a second spike 102, 202, 302, 402, 502, 602 of the respective intravenous tubing set 100, 200, 300, 400, 500, 600, either simultaneously with or subsequently to the coupling of the first infusion vial 156 and administration of the contents of the first infusion vial 156. For instance, the second spike 102, 202, 302, 402, 502, 602 may be on a separate line of tubing 104, 204, 304, 404, 504, 604 as the first spike 102, 202, 302, 402, 502, 602. Alternatively, the line of tubing 104, 204, 304, 404, 504, 604 may be branched and in fluid communication with both spikes 102, 202, 302, 402, 502, 602.

In embodiments, any of the spikes 102, 202, 302, 402, 502, 602 may optionally be interchanged with another spike 102, 202, 302, 402, 502, 602 prior to the coupling of the second infusion vial to the respective intravenous tubing set 100, 200, 300, 400, 500, 600, either with or without the respective lengths of tubing 104, 204, 304, 404, 504, 604. It is also noted that the spike 112 for the intravenous bag of solution may similarly be interchanged with another spike 112, without departing from the scope of the present disclosure.

Although embodiments of the present disclosure are directed to one or more of the spikes 102, 202, 302, 402, 502, 602 being vented, such as where the particular spike 102, 202, 302, 402, 502, 602 are inserted into a stopper 158 of an infusion vial 156, it should be understood that non-vented spikes may be used where an infusion bag with non-rigid sides is used, without departing from the scope of the present disclosure.

Although the method or process 700 is described as being usable for two portions of a dose for a pharmaceutical composition, it should be understood that the present disclosure is not limited to a two-portion dose. Rather, the method or process 700 (and the intravenous tubing set 100, 200, 300, 400, 500, 600 usable for the method or process 700) may be modified to be usable for a pharmaceutical composition with any number of portions that make up a dose of the pharmaceutical composition, without departing from the scope of the present disclosure.

In this regard, advantages of the present disclosure include, but are not limited to, a system and method for administering a pharmaceutical composition with an intravenous tubing set. The intravenous tubing set includes a first vented spike on a first line for a pharmaceutical composition and a second non-vented spike on a second line for a solution. The first line and the second line are in fluid communication with a dual port chamber, which is in fluid communication with a coupler that is able to engage an instrument attached to a patient at an intravenous site.

Advantages of the present disclosure also include, but are not limited to, are also directed to an intravenous tubing set capable of administering a dose of pharmaceutical composition that is separated into different infusion vials. The intravenous tubing set includes a vented spike that is insertable into the different infusion vials. The vented spike does not need to be changed between infusion vials due to clogging of a filter within the vented spike, or the filter being made wet, by the pharmaceutical composition from the first infusion vial. The vented spike is insertable into the stopper of the infusion vial without unseating or dislodging the stopper, which may cause leakage from the infusion vial (i.e., due to the suspension of the infusion vial for purposes of gravity feeding through the intravenous tubing set).

The phrases “at least one,” “one or more,” and “and/or,” as used herein, are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Unless otherwise indicated, all numbers expressing quantities, dimensions, conditions, and so forth used in the specification and claims are to be understood as being modified in all instances by the term “about.”

The use of “substantially” in the present disclosure, when referring to a measurable quantity (e.g., a diameter or other distance) and used for purposes of comparison, is intended to mean within 5% of the comparative quantity. The terms “substantially similar to,” “substantially the same as,” and “substantially equal to,” as used herein, should be interpreted as if explicitly reciting and encompassing the special case in which the items of comparison are “similar to,” “the same as” and “equal to,” respectively.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The term “a” or “an” entity, as used herein, refers to one or more of that entity. As such, the terms “a” (or “an”), “one or more” and “at least one” can be used interchangeably herein.

The use of “including,” “comprising,” or “having” and variations thereof herein is meant to encompass the items listed thereafter and equivalents thereof as well as additional items. Accordingly, the terms “including,” “comprising,” or “having” and variations thereof can be used interchangeably herein. The use of “engaged with” and variations thereof herein is meant to encompass any direct or indirect connections between components.

It shall be understood that the term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials, or acts and the equivalents thereof shall include all those described in the summary of the disclosure, brief description of the drawings, detailed description, abstract, and claims themselves.

All external references are hereby incorporated by reference in their entirety whether explicitly stated or not.

It is to be appreciated that any embodiment, feature, or aspect described herein can be claimed in combination with any other embodiment(s), feature(s), or aspect(s) as described herein, regardless of whether the features or aspects come from the same described embodiment. For example, any one or more aspects described herein can be combined with any other one or more aspects described herein. In addition, any one or more features described herein can be combined with any other one or more features described herein. Further, any one or more embodiments described herein can be combined with any other one or more embodiments described herein.

While various embodiments of the present disclosure have been described in detail, it is apparent that modifications and alterations of those embodiments will occur to those skilled in the art. However, it is to be understood that such modifications and alterations are within the scope and spirit of the present disclosure, as set forth in the following claims. Further, the disclosure described herein is capable of other embodiments and of being practiced or of being carried out in various ways. It is to be understood that the phraseology and terminology used herein is for the purpose of description and should not be regarded as limiting.

What is claimed is:

1. An intravenous tubing set, comprising:
a vented spike operable to be inserted into a stopper of an infusion vial;
a first line of tubing having a first end in fluid communication with the vented spike;
a non-vented spike operable to be inserted into an intravenous bag of solution;
a second line of tubing having a first end in fluid communication with the non-vented spike; and
a dual port chamber, wherein a second opposite end of the first line of tubing and a second opposite end of the second line of tubing are each in fluid communication with a first end of the dual port chamber.

2. The intravenous tubing set of claim 1, further comprising:
a first clamp operable to control a fluid flow through the first line of tubing; and
a second clamp operable to control a fluid flow through the second line of tubing.

3. The intravenous tubing set of claim 1, wherein the first line of tubing is in fluid communication with a coupler, wherein the coupler is able to engage the vented spike.

4. The intravenous tubing set of claim 3, wherein the coupler able to engage the vented spike includes a coupling mechanism comprising a spinlock or a Luer lock.

5. The intravenous tubing set of claim 3, wherein the coupler able to engage the vented spike includes a cap operable to cover a distal end of the coupler.

6. The intravenous tubing set of claim 1, further comprising:
a third line of tubing in fluid communication with a second end of the dual port chamber and in fluid communication with a coupler, wherein the coupler is able to engage an instrument attached to a patient at an intravenous site.

7. The intravenous tubing set of claim 6, further comprising:
a clamp operable to control a fluid flow through the third line of tubing.

8. The intravenous tubing set of claim 6, wherein the coupler able to engage the instrument includes a coupling mechanism comprising a spinlock or a Luer lock.

9. The intravenous tubing set of claim 6, wherein the coupler able to engage the instrument includes a cap operable to cover a distal end of the coupler.

10. The intravenous tubing set of claim 6, further comprising:
an additional connector separating the third line of tubing into a first section and a second section,
wherein the first section of the third line of tubing is in fluid communication with the second end of the dual port chamber and in fluid communication with a first end of the additional connector, and
wherein the second section of the third line of tubing is in fluid communication with a second end of the additional connector and in fluid communication with the additional connector.

11. The intravenous tubing set of claim 10, wherein a first end of the additional connector includes a first branch and a second branch, and wherein the first section of the third line of tubing is in fluid communication with the first branch of the additional connector.

12. The intravenous tubing set of claim 11, wherein the second branch of the additional connector is operable to provide access to the second section of the third line of tubing.

13. The intravenous tubing set of claim 11, wherein the second end of the additional connector and the second branch of the first end of the additional connector are substantially collinear along a longitudinal axis through the additional connector, and wherein the first branch of the first end of the additional connector is set at an angle to the longitudinal axis through the additional connector.

14. The intravenous tubing set of claim 1, wherein the dual port chamber includes a cap at the first end.

15. The intravenous tubing set of claim 14, wherein the first line of tubing and the second line of tubing are separately in fluid communication with the cap.

16. The intravenous tubing set of claim 1, wherein the dual port chamber includes a port at a second end opposite to the first end of the dual port chamber.

17. An intravenous tubing set, comprising:
tubing comprising a plurality of lines;
a non-vented spike coupled to a first line of the plurality of lines;
at least a second vented spike coupled to at least a second line of the plurality of lines; and
a dual port chamber, wherein the first line and the at least a second line of the plurality of lines are in fluid communication with a stopper at a first end of the dual port chamber.

18. The intravenous tubing set of claim 17, wherein the plurality of lines comprises:
a first line of tubing having a first end in fluid communication with the at least a second vented spike; and
a second line of tubing having a first end in fluid communication with the non-vented spike,
wherein a second opposite end of the first line of tubing and a second opposite end of the second line of tubing are each in fluid communication with the first end of the dual port chamber.

19. A method for administering a pharmaceutical composition, comprising:
coupling a first infusion vial and an intravenous bag of solution to an intravenous tubing set to administer a first portion of the pharmaceutical composition, the intravenous tubing set comprising:
a vented spike operable to be inserted into a stopper of the first infusion vial;
a first line of tubing having a first end in fluid communication with the vented spike;
a non-vented spike operable to be inserted into the intravenous bag of solution;
a second line of tubing having a first end in fluid communication with the non-vented spike; and
a dual port chamber, wherein a second opposite end of the first line of tubing and a second opposite end of the second line of tubing are in fluid communication with a first end of the dual port chamber;
uncoupling the first infusion vial from the intravenous tubing set; and
coupling a second infusion vial to the intravenous tubing set to administer a second portion of the pharmaceutical composition.

20. The method of claim 19, wherein the vented spike is interchangeable with a second vented spike prior to the coupling of the second infusion vial to the intravenous tubing set, wherein the second vented spike is operable to be inserted into a stopper of the second infusion vial.
